(19) United States
(12) Patent Application Publication
(10) Pub. No.: US 2006/0106233 A1
(43) Pub. Date: May 18, 2006

(54) Process for the preparation of disubstituted acetylenes bearing heteroaromatic and heterobicyclic groups

(75) Inventors: Bobba Venkata Siva Kumar, Navi Mumbai (IN); Shekhar Bhaskar Bhirud, Navi Mumbai (IN); Batchu Chandrasekhar, Navi Mumbai (IN); Changdev Namdev Raut, Navi Mumbai (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/155,847

(22) Filed: Jun. 17, 2005

Related U.S. Application Data
(60) Provisional application No. 60/580,495, filed on Jun. 17, 2004.

(51) Int. Cl. C07D 335/06; C07D 215/60

(52) U.S. Cl. 549/23; 546/153; 549/402

(57) ABSTRACT

A process for the preparation of a disubstituted acetylene bearing heteroaromatic and heterobicyclic groups of formula I is provided 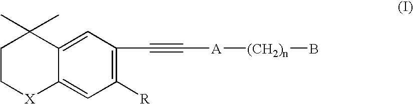
wherein X is S, O, or NR¹ wherein R¹ is hydrogen or a C₁-C₆ straight or branched alkyl group; R is hydrogen or a C₁-C₆ straight or branched alkyl group; A is a substituted or unsubstituted pyridinyl, thienyl, furyl, pyridazinyl, pyrimidinyl or pyrazinyl group; n is 0-4; and B is H, —COOH, —CH₂OH, —CHO or a C₁-C₆ alkyl acetal derivative, —COR² or a C₁-C₆ alkyl ketal derivative where R² is —(CH₂)ₘCH₃ where m is 0-4 or COOR³ wherein R³ is a straight or branched C₁-C₃₀ alkyl group, a substituted or unsubstituted C₆-C₃₀ aromatic group, a substituted or unsubstituted C₃-C₃₀ cycloalkyl, a substituted or unsubstituted C₃-C₃₀ cycloalkylalkyl, a substituted or unsubstituted C₃-C₃₀ cycloalkenyl, a substituted or unsubstituted C₅-C₃₀ aryl, a substituted or unsubstituted C₅-C₃₀ arylalkyl, a substituted or unsubstituted C₅-C₃₀ heteroaryl, a substituted or unsubstituted C₃-C₃₀ heterocyclic ring, a substituted or unsubstituted C₄-C₃₀ heterocyclylalkyl, a substituted or unsubstituted C₆-C₃₀ heteroarylalkyl, the process comprising a Sonogashira coupling reaction between a compound of formula II 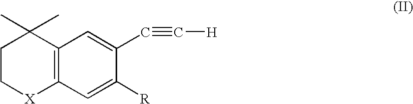 wherein X and R have the aforestated meanings, with a compound of formula III
X′-A-(CH₂)ₙ—B (III) wherein X′ is a halogen and A, n and B have the aforestated meanings, in the presence of a base and a transition metal catalyst and in a polar aprotic solvent.

### CROSS-REFERENCE TO RELATED APPLICATIONS

This application claims the benefit under 35 U.S.C. §119 to Provisional Application No. 60/580,495, filed Jun. 17, 2004 and entitled “PROCESS FOR THE PREPARATION OF TAZAROTENE”, the contents of which are incorporated by reference herein.

### BACKGROUND OF THE INVENTION

1. Technical Field

The present invention generally relates to an improved process for the preparation of disubstituted acetylenes bearing heteroaromatic and heterobicyclic groups. More specifically, the present invention generally relates to a process for the preparation of disubstituted acetylenes bearing heteroaromatic and heterobicyclic groups employing a Sonogashira coupling reaction.

2. Description of the Related Art

The present invention is directed towards an improved process for the preparation of disubstituted acetylenes bearing heteroaromatic and heterobicyclic groups such as tazarotene (also known as ethyl-6-[2-(4,4-dimethylthiochroman-6-yl)-ethynyl]) of the formula: 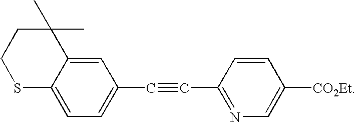
Tazarotene is a member of the acetylenic class of retinoids and is a prodrug that is converted to its active drug form, known as AGN 190299, in most biological systems by rapid deesterificaion of the cognate carboxylic acid of tazarotene. AGN 190299 binds to all three members of the retinoic acid receptor (RAR) family: RARα, RARβ, RARγ. AGN 190299 shows relative selectivity for the RARβ and RARγ and may modify gene expression. Tazarotene is ordinarily used in the treatment of psoriasis and is commercially available under the trade name Tazorac®.

It would be desirable to provide an improved process for preparing disubstituted acetylenes bearing heteroaromatic and heterobicyclic groups such as tazarotene in a convenient and cost efficient manner and on a commercial scale.

### SUMMARY OF THE INVENTION

In one embodiment of the present invention, a process for the preparation of a disubstituted acetylene bearing heteroaromatic and heterobicyclic groups of formula I 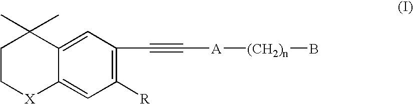
wherein X, R, A, n and B are as defined herein is provided, the process comprising a Sonogashira coupling reaction between a compound of formula II 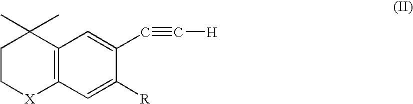
with a compound of formula III
X′-A-(CH₂)ₙ—B (III)
wherein X′ is as defined herein in the presence of a base and a transition metal catalyst and in a polar aprotic solvent.

In another embodiment of the present invention, the process further comprises (a) adding an inorganic acid to the reaction mixture following the Sonogashira coupling reaction to provide a salt of the disubstituted acetylene; (b) adding an inorganic base to the salt in a second solvent and (c) isolating the disubstituted acetylene from the second solvent.

The advantages of the present invention include at least:

1. By carrying out the Sonogashira coupling of the two intermediates of formula II and III in a polar aprotic solvent, for example, dimethyl sulfoxide (DMSO), dimethyl formamide (DMF) and dimethyl acetamide (DMA), the reaction time of coupling the intermediates may be advantageously reduced while controlling the formation of impurities thereby providing a product with a higher purity level.

2. The purification of the disubstituted acetylene compounds (e.g., tazarotene) herein by forming a corresponding salt in situ and regenerating the disubstituted acetylene compound simplifies the procedure of conventional purification by flash or preparative chromatography, as well as improving the overall yield.

### DETAILED DESCRIPTION OF THE PREFERRED EMBODIMENTS

In one aspect of the present invention, a process for preparing a disubstituted acetylene bearing heteroaromatic and heterobicyclic groups such as tazarotene is provided employing a Sonogashira coupling reaction. In one embodiment, the process for the preparation of a disubstituted acetylene bearing heteroaromatic and heterobicyclic groups of formula I 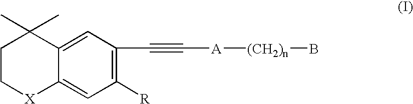
wherein X is S, O, or NR¹ wherein R¹ is hydrogen or a C₁-C₆ straight or branched alkyl group; R is hydrogen or a C₁-C₆ straight or branched alkyl group; A is a substituted or unsubstituted pyridinyl, thienyl, furyl, pyridazinyl, pyrimidinyl or pyrazinyl group; n is 0-4; and B is H, —COOH or a pharmaceutically acceptable salt thereof, or an ester thereof with, for example, a saturated aliphatic alcohol of ten or fewer carbon atoms, or with a cyclic or saturated aliphatic cyclic alcohol of 5 to 10 carbon atoms, or with a phenol or a lower alkylphenol, or an amide or a mono or di-substituted amide thereof, the subtituents on the amide being, for example, a saturated aliphatic radical containing about 10 or fewer carbon atoms, a cyclic or saturated aliphatic cyclic radical of 5 to about 10 carbon atoms, a phenyl or lower alkylphenyl radical, —CH₂OH, —CHO or a C₁-C₆ alkyl acetal derivative, —COR² or a C₁-C₆ alkyl ketal derivative wherein R² is —(CH₂)ₘCH₃ wherein m is 0-4 or COOR³ wherein R³ is a straight or branched C₁-C₃₀ alkyl group, a substituted or unsubstituted C₆-C₃₀ aromatic group, a substituted or unsubstituted C₃-C₃₀ cycloalkyl, a substituted or unsubstituted C₃-C₃₀ cycloalkylalkyl, a substituted or unsubstituted C₃-C₃₀ cycloalkenyl, a substituted or unsubstituted C₅-C₃₀ aryl, a substituted or unsubstituted C₅-C₃₀ arylalkyl, a substituted or unsubstituted C₅-C₃₀ heteroaryl, a substituted or unsubstituted C₃-C₃₀ heterocyclic ring, a substituted or unsubstituted C₄-C₃₀ heterocyclylalkyl, a substituted or unsubstituted C₆-C₃₀ heteroarylalkyl; includes at least a Sonogashira coupling reaction between a compound of formula II 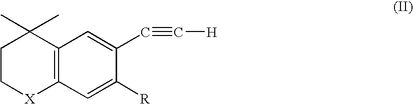
wherein X and R have the aforestated meanings, with a compound of formula III
X′-A-(CH₂)ₙ—B (III)
wherein X′ is a halogen such as Cl, Br or I, and A, n and B have the aforestated meanings, in the presence of a base and a transition metal catalyst and in a polar aprotic solvent. The above-described alkyl groups, aromatic groups, cycloalkyls, cycloalkylalkyls, cycloalkenyls, aryls, arylalkyls, heteroaryls, heterocyclic rings, heterocyclylalkyls, heteroarylalkyls may each be substituted with moieties such as alkyl moieties, nitrogen-containing moieties (e.g., amino, amido, etc.), oxygen-containing moieties (e.g., hydroxyl, carboxyl, etc.), halogens, sulfur-containing moieties (e.g., thiol, sulfonyl, etc.) and the like.

Amide as used herein has the meaning classically accorded that term in organic chemistry. For example, it includes the unsubstituted amides and all aliphatic and aromatic mono- and di-substituted amides. Preferred amides are the mono- and di-substituted amides derived from a saturated aliphatic radical of 1 to about 10 carbon atoms or a cyclic or saturated aliphatic-cyclic radical of 5 to about 10 carbon atoms. Particularly preferred amides are those derived from lower alkyl amines. Also preferred are mono- and di-substituted amides derived from a phenyl or lower alkylphenyl amine. Unsubstituted amides are also contemplated.

Acetals and ketals include the radicals of the formula —CK wherein K is (—OR⁴)₂ wherein R⁴ is lower straight or branched alkyl of 1 to 5 carbon atoms. Also, K may be —OR⁵O— wherein R⁵ is lower alkyl of 1 to 5 carbon atoms, straight chain or branched.

Representative examples of the compounds of formula I that can be obtained by the process of the present invention include the following ethyl 6-(2-(4,4-dimethylthiochroman-6-yl)ethynyl)nicotinate; 6-(2-(4,4-dimethylthiochroman-6-yl)ethynyl)nicotinic acid; 6-(2-(4,4-dimethylchroman-6-yl)ethynyl)nicotinic acid; ethyl 6-(2-(4,4-dimethylchroman-6-yl)-ethynyl)nicotinate; ethyl 6-(2-(4,4,7-trimethylthiochroman-6-yl)ethynyl)nicotinate; ethyl 6-(2-(4,4-dimethyl-1,2,3,4-tetrahydroquinolin-6-yl)-ethynyl)nicotinate; ethyl 5-(2-(4,4-dimethylthiochroman-6-yl)ethynyl)-thiophene-2-carboxylate. 6-(2-(4,4-dimethylthiochroman-6-yl)ethynyl)-3-pyridylmethanol; and 2-(2-(4,4-dimethylthiochroman-6-yl)-ethynyl)-5-pyridinecarboxaldehyde. The preferred compounds formed by the process of this invention are those where the ethynyl group and the B group are attached to the 2 and 5 positions respectively of a pyridine ring.

The preferred compounds of formula II are those where X is S and R is hydrogen, i.e., 4,4-dimethyl-6-ethynylthiochroman. Preferred compounds of formula III are those where n is 0, B is —COOH, an alkali metal salt or organic amine salt, or —COOR³ wherein R³ is a straight or branched C₁-C₆ alkyl group, a substituted or unsubstituted C₆-C₃₀ aromatic group, or a substituted or unsubstituted C₃-C₃₀ cycloalkyl. Compounds of formula II and III are known and can be obtained by processes well known in the art. See, e.g., Examples 4 and 5 of U.S. Pat. No. 5,602,130, the contents of which are incorporated by reference herein.

A suitable base for use herein may be, for example, an organic base such as a primary, secondary or tertiary amine. Representative examples of such amines include, but are not limited to, triethylamine, tributylamine, diisopropylethylamine, diethylamine, N-methylmorpholine, pyridine, 4-(N,N-dimethylamino)pyridine, N,N-dimethylaniline, N,N-diethylaniline, 1,5-diazabicyclo[4.3.0]nona-5-ene, 1,4-diazabicyclo[2.2.2]octane (DABCO), 1,8-diazabicyclo[5.4.0]undec-7-ene (DBU) and the like and mixtures thereof. Alternatively, an inorganic base may be used and include, but are not limited to, alkali metal carbonates such as lithium carbonate, sodium carbonate, potassium carbonate and the like; alkali metal bicarbonates such as lithium bicarbonate, sodium bicarbonate, potassium bicarbonate and the like; alkali metal hydrides such as lithium hydride, sodium hydride, potassium hydride and the like; alkali metal hydroxides such as lithium hydroxide, sodium hydroxide, potassium hydroxide and the like; alkali metal alkoxides such as lithium methoxide, sodium methoxide, sodium ethoxide, potassium t-butoxide and the like; and mixtures thereof. The organic amines (particularly triethylamine) are preferred.

The transition metal catalyst may be in the form of a salt or a complex with organic ligands. Particularly suitable metal catalysts are, for example, the Group VIII metals, such as Pd(0) complexes or a Pd(II) salt. However, the palladium catalyst used is not particularly limited provided that it is usually used for the Sonogashira coupling reaction. The ligands may be selected from, for example, phosphorus-containing ligands, such as triphenylphosphine (PPh₃) and 1,2-bis(diphenyl-phosphino)ethane (dppe). Non-limiting examples of the transition metal catalysts include palladium salts such as palladium acetate, palladium chloride or palladium carbonate; and palladium complexes such as bis(triphenylphosphine) palladium (II) chloride (Pd[P(C₆H₅)₃]₂Cl₂) and palladium (0) based catalysts, such as Pd Cl₂(RCN)₂, wherein R is phenyl or methyl and mixtures thereof.

The reaction is advantageously carried out in a polar aprotic solvent. Suitable polar aprotic solvents include, but are not limited to, nitrites such as acetonitrile, isobutyronitrile and the like; dioxane, amides such as formamide, dimethylformamide, dimethylacetamide, hexamethylphosphoric triamide and the like; sulfoxides such as dimethyl sulfoxide, sulfolane and the like; as well as other polar aprotic solvents and mixtures thereof. Preferably, the polar aprotic solvent is an amide or sulfoxide with dimethyl sulfoxide, dimethyl form amide and dimethyl acetamide being more preferred. In a preferred embodiment of the present invention, the polar aprotic solvent is dimethyl sulfoxide. Generally, the amount of polar aprotic solvent employed in the coupling reaction can range from about 5 volumes to about 15 volumes and preferably from about 7 volumes to about 10 volumes.

The reaction can be carried out in the presence of a cuprous halide. The cuprous halide for use herein includes, but is not limited to, cuprous fluoride, cuprous chloride, cuprous bromide, cuprous iodide and the like and mixtures thereof. In a preferred embodiment of the process of the present invention, the cuprous halide is cuprous iodide.

The reaction temperature and time period for coupling the foregoing intermediates of formula II and III will ordinarily depend on the starting compounds, the base and the solvent employed in the reaction. Generally, the reaction can be carried out at a temperature of from about 20° C. to about 200° C. for about 5 minutes to about 48 hours and preferably from about 15 minutes to about 24 hours. The reaction is advantageously conducted under an inert atmosphere such as nitrogen. Generally, to prepare a disubstituted acetylene such as tazarotene, a solution containing the transition metal catalyst and solvent may first be heated to a temperature ranging from about 130° C. to about 150° C. and preferably from about 140° C. to about 145° C. under a nitrogen atmosphere. As one skilled in the art will readily appreciate, the transition metal catalyst may be formed in situ by adding the salt with the organic ligands to the solution. Next, a solution of a compound of formula II, e.g., 4,4-dimethyl-6-ethynylthiochroman, a compound of formula III, e.g., ethyl-6-chloro-3-nicotinate, base (e.g., triethanolamine) and cuprous halide are mixed separately and then added to the solution containing the transitional metal catalyst and solvent. The reaction mixture may then be heated to a temperature ranging from about 80° C. to about 100° C., and preferably to a temperature from about 95° C. to about 100° C., and stirred for about 2 to about 4 hours, and preferably about 3 hours.

In one embodiment, the process of the present invention includes coupling intermediates 4,4-dimethyl-6-ethynylthiochroman and ethyl-6-chloro-3-nicotinate (also known as ethyl-6-chloropyridine-3-carboxylate) in the presence of a base and a transition metal catalyst and in a polar aprotic solvent.

Following the completion of the Sonogashira coupling reaction, the disubstituted acetylene compounds thus obtained may be purified. For example, an inorganic acid may be added to the reaction mixture prior to any isolation or following isolation after completion of the coupling reaction to provide a salt of the disubstituted acetylene compound. Examples of suitable inorganic acids include, but are not limited to, hydrobromic acid, hydrochloric acid, sulfuric acid, perchloric acid, phosphoric acid and the like, as well as solutions of the inorganic acid. e.g., in an acetate such as ethyl acetate, with hydrochloric acid being preferred. By adding the inorganic acid to the reaction mixture, a salt of the compound, e.g., tazarotene, is advantageously formed. If desired, the inorganic acid can be added as a solution further containing a suitable solvent such as, for example, ethyl acetate. The salt obtained can then be dissolved in a second solvent and an inorganic base may be added such that the disubstituted acetylene compound can be isolated by conventional techniques. This allows for a higher yield of the resulting disubstituted acetylene such as tazarotene from the salt compound, e.g., a yield of at least about 65% and preferably at least about 80%, as well as a high purity level, e.g., a purity of at least about 95% preferably at least about 98% and more preferably at least about 99.5%.

The second solvent for use herein includes, but is not limited to, aromatic hydrocarbon solvents such as toluene, xylene and the like; ketones such as methyl isobutyl ketone and the like; acetates such as methyl acetate, t-butyl acetate and the like, alcohols such as methanol, ethanol, N-butanol and the like and mixtures thereof.

A suitable inorganic base for use herein includes, but is not limited to, alkali metal carbonates such as potassium carbonate, sodium carbonate, and the like; alkali metal bicarbonates such as potassium bicarbonate and the like; alkali metal hydroxides such as sodium hydroxide, potassium hydroxide and the like and mixtures thereof.

The following examples are provided to enable one skilled in the art to practice the invention and are merely illustrative of the invention. The examples should not be read as limiting the scope of the invention as defined in the claims.

### EXAMPLE 1

### Step I: Preparation of phenyl-3-methylbut-2-enyl sulfide

Into a 5 L 4-neck round bottom flask, methanol (1400 ml) and thiophenol (200 g) were added under stirring at a temperature ranging from about 25° C. to about 35° C. Sodium hydroxide (powder LR grade) (73.60 g) and methanol (100 ml) were added to the mixture under stirring. The reaction mixture was left under a nitrogen atmosphere and stirred at room temperature (about 25° C. to about 30° C.) for an hour. Next, 1-bromo-3-methyl-2-butene (274 gm) was added to the reaction mixture and it was observed that the temperature rose to about 40° C. The reaction mixture was heated to reflux and maintained for about 12 hours. After completion of the reaction as determined by HPLC, the methanol was distilled out from the reaction mixture under vacuum at a temperature below 60° C. Ethylene dichloride (1500 ml) and water (1000 ml) were added to the residue. The organic layer was separated and washed with a 5% sodium hydroxide (600 ml) solution, and then water (3×600 ml) until the pH was about 7. The organic layer was then washed with a brine solution (700 ml). The ethylene dichloride was distilled out until the moisture content was less than 0.1%.

### Step II: Preparation of 4,4-dimethylthiochroman

Into a 5 L 4-neck round bottom flask, ethylene dichloride (1500 ml) was added to the phenyl-3-methylbut-2-enyl sulfide obtained in step I. Phosphorous pentoxide (200 gm) was added to the reaction mixture at a temperature ranging from about 25° C. to about 35° C. under stirring. Next, ortho phosphoric acid (174 ml) was added carefully under nitrogen. The reaction mixture was heated to reflux, a temperature of about 80° C. to about 90° C. and maintained at that temperature for about 12 hours. After completion of the reaction as determined by HPLC, the reaction mass was cooled to a temperature ranging from about 25° C. to about 35° C. and water (2000 ml) was slowly added to the reaction mass. The organic layer was separated, and the aqueous layer was extracted with ethylene dichloride (2 L×2). The organic layers were combined and washed with saturated sodium bicarbonate solution (2 L×2) and water (1.5 L×2) until the pH was about 7. This was followed by a washing with a brine solution (1.5 L). The ethylene dichloride layer was distilled out under reduced pressure below a temperature of about 70° C. until the moisture content was less than 0.1%. Ethylene dichloride (2 L) was added to the residue and taken for the next step without further purification

### Step III: Preparation of 4,4-dimethyl-6-acetylthiochroman

Into a 5 L 4-neck round bottom flask, ethylene dichloride (2 L) was added to the 4,4-dimethylthiochroman obtained in step II. The contents were stirred and cooled to a temperature of about −10° C. Aluminum chloride (252 g) was slowly added to the reaction mixture. Acetyl chloride (152.7 g) was added at a temperature ranging from about −10° C. to about −5° C. over about 1.5 hours. After the addition, the reaction mixture was maintained at a temperature ranging from about −5° C. to about 0° C. for about 2 hours. The reaction was monitored by TLC. If the reaction is incomplete as determined by TLC, bring the reaction mixture to a temperature ranging from about 25° C. to about 35° C. under stirring for about 4 hours. The reaction mixture was quenched with ice (4.87 kg) and hydrochloric acid (1.63 L), and the reaction mass was stirred for about 30 minutes. Ethylene dichloride (2.5 L) was added to the reaction mass and the layers were separated. The aqueous layer was extracted with methylene dichloride (2×2 L). The organic layers were combined and washed with 5% sodium bicarbonate solution (2×2 L) and water (2×2 L) until the pH was about 7. This was followed by a washing with brine (1.5 L). The ethylene dichloride and methylene dichloride layer were distilled out under reduced pressure until the moisture content was less than about 0.1%. There was a residual volume of about 3 L.

### Step IV: Preparation of 3-[4,4-dimethylthiochroman-6-yl]-3-chloro-2-propene-1-al

Into a 500 ml 4-necked round bottom flask fitted with a mechanical stirrer and a reflux condenser, 6-acetyl-4,4-dimethylthio-chroman (22 g) and dimethylformamide (38 ml) were added at a temperature in the range of from about 35° C. to about 95° C. under stirring. The reaction mixture was then cooled to a temperature in the range of from about −5° C. to about 0° C. Phosphorus oxychloride (17.2 g) was added to the reaction mixture dropwise over about 30 minutes. Following the addition of the phosphorous oxychloride, the reaction mixture was maintained at a temperature in the range of from about 10° C. to about 15° C. for about 8 hours to about 10 hours. After completion of the reaction as determined by TLC, the reaction mixture was added to cold water (100 ml) at a temperature of from about 0° C. to about 5° C. containing sodium acetate (25 g). The aqueous layer was extracted with dichloromethane (DCM) (200 ml×3). The organic layer was washed with demineralized water (100 ml×3) until it became neutral.

The DCM layer was concentrated on a rotavapor bath at a temperature in the range of from about 25° C. to about 30° C. under plant vacuum until no more drops were observed. The resulting residual oil was purified by flash chromatography with petroleum ether and ethyl acetate (9:1 mixture) resulting in a pale yellow oil, weighing about 22 g, yield of about 82%, purity of about 98% (HPLC). The IR (neat) showed the following stretching 2900 cm⁻¹ (C—H str), 2750 cm⁻¹ (C—H str), 1690 cm⁻¹ (—C═O str), 1620 cm⁻¹ (—C═C-str), 760 cm⁻¹ (—C═C—Cl str). The ¹H-NMR (CDCl₃) using TMS as internal standard showed the following signals at δ 1.35 (6H,s) 1.92-1.98 (2H,m), 3.02-3.08 (2H,m), 5.5 (1H,s), 7.13 (1H,d 8.6 Hz), 7.58 (1H,dd,J 8.6 Hz,2H), 7.99 (1H,d,J 2 Hz), 8.9 (s,1H). The CI mass showed m/z 266 (M+).

### Step V: Preparation of 4,4-dimethyl-6-ethynylthiochroman

Into a 250 ml 4-necked round bottom flask fitted with a mechanical stirrer and reflux condenser, water (41.3 ml) and sodium hydroxide (5.22 g, 0.1305M) were added and heated to a temperature in the range of from about 80° C. to about 90° C. The reaction mixture was stirred, and a solution of 3-[4,4-dimethylthiochroman-6-yl]-3-chloro-2-propene-1-al (3.0 gm, 0.0113 M) was added dropwise in 1,4-dioxane (52.2 ml) under vigorous stirring. The reaction mixture was maintained at a temperature in the range of from about 80° C. to about 90° C. for about 2 hours. After completion of the reaction as determined by TLC, the solvents were distilled off and the product was extracted with ether (15 ml×3). The ether layer was washed with brine (15 ml×3). The organic layer was dried over sodium sulfate, and the solvent was distilled off to get an oily residue. The resulting crude oil was distilled under high vacuum and the vapors were collected at a temperature of about 126° C./0.2 mm as the main product. The main fraction appeared as a red viscous oil, which upon standing crystallized. The product showed a net weight of about 2.00 g, a yield of about 87.68%; a m.p. in the range of from about 69° C. to about 72° C., and a purity of about 98% (HPLC). The IR (neat) showed the following absorptions: 3200 cm⁻¹ (C—H-str), 2950 cm⁻¹ (—C═C—H str), 2100 cm⁻¹ (—C═C—). The ¹H-NMR (CDCl₃), TMS as internal standard showed the following signals δ 1.35 (6H,s), 1.92-1.98 (2H,m), 3.02-3.08 (3H,m), 7.13 (1H,d 8.6 Hz), 7.58 (1H,dd,J 8.6 Hz,2 Hz), 7.99 (1H,d,J 2 Hz). The CI/MS showed m/z 202 (M+).

### EXAMPLE 2

### Step I: Preparation of Tazarotene Hydrochloride Salt

Into a 2 L 4-neck round bottom flask, dimethyl sulfoxide (700 ml), palladium chloride (3.83 g), and triphenyl phosphine (14.06 g) were added under stirring under a nitrogen atmosphere at room temperature and the temperature was slowly raised to about 145° C. The solution became clear at a temperature of about 145° C. after about 10 minutes. Then the solution was slowly cooled to room temperature over about 45 minutes. In a separate 2 L 4-neck round bottom flask, ethyl-6-chloro-3-nicotinate (96.40 g), 4,4-dimethy-6-ethynylthiochroman (100 g) obtained in Example 1, cuprous iodide (6.5 g), and triethanolamine (TEA) (165 g) were added at room temperature under a nitrogen atmosphere. The reaction mixture was stirred for about 2 to about 5 minutes and the contents from the other round bottom flask (now containing bis(triphenylphosphine) palladium (II) chloride formed in situ) were added to the reaction mixture. The temperature was slowly raised to a temperature of about 98° C. and maintained for about three hours. After the reaction was completed as determined by TLC, the reaction mixture was cooled to a temperature of about 20° C. to form tazarotene. The reaction mixture was then filtered, and the resulting cake was washed with DMSO (20 ml). All the filtrate was combined, and ethyl acetate (1 L) was added to the filtrate. The solution was washed with water (3×400 ml). The organic layer was separated, and the ethyl acetate was distilled out completely (KFR<0.2%) under vacuum. An ethyl acetate HCl solution (1000 ml) was added to the residue within 15 minutes. The reaction mixture was maintained for 2 hours at room temperature to form tazarotene hydrochloride salt. The tazarotene hydrochloride solid was filtered and washed with ethyl acetate (2×150 ml). The solid was dried at room temperature for about 6 hours (to remove excess HCl gas). Tazarotene hydrochloride salt appears as a yellow solid after drying. The solid weighed about 140 g. Yield=81%, m.p. 112-114° C., purity 99.6% (by HPLC).

The IR (KBr) spectrum showed stretching at 2204 cm⁻¹ and 1720 cm⁻¹. The ¹H NMR (CDCl₃) showed signals at δ 9.2 (s,1H), 8.6 (1H,d), 7.8 (d,2H), 7.4(d,1H), 4.4(q,2H), 3.1(dd,2H), 2.0(dd,2H), 1.5-1.6(t,3H), 1.2-1.4(s,6H). The CI Mass showed M+. m/z 352.

### Step II: Preparation of Tazarotene

Into a 5 L 4-neck round bottom flask, ethyl acetate (1500 ml) and a saturated solution of sodium bicarbonate (NaHCO₃) (1500 ml) were added under stirring at room temperature. The tazarotene hydrochloride salt (140 gm) obtained in step I was slowly added to the reaction mixture. The reaction mixture was stirred for about 2 hours to get a clear solution. The pH should be about 7.5 to about 8, and if not, then further saturated NaHCO₃ solution (500 ml) was added until the pH was about 7.5 to about 8. The organic and the aqueous layer were then separated. The organic layer was washed with water (400 ml×3) and followed by washing with a saturated NaCl solution (200 ml). The organic layer was decolorized with charcoal (10 g) at room temperature by stirring for about 1 hour. The reaction mixture was filtered through a Hyflow and washed with ethyl acetate (200 ml). The ethyl acetate was distilled out under vacuum at a temperature ranging from about 40° C. to about 45° C. until no more drops were observed. Ethyl acetate (200 ml) was added at a temperature of about 40° C., and the reaction mixture was heated to a temperature ranging from about 60° C. to about 65° C. to get a clear solution. The reaction mixture was gradually cooled to room temperature over about 2 hours. The reaction mixture was then cooled to a temperature ranging from about −5° C. to about 0° C. and stirred for about 1 hour. The reaction mixture was filtered and washed with chilled ethyl acetate (120 ml). The reaction mixture was dried at a temperature ranging from about 40° C. to about 45° C. The product appeared as an off-white to yellowish-white solid. Net wt. of about 94 g, yield 55%, purity 99.5% (HPLC).

The IR (KBr) spectrum showed stretching at 2900 cm⁻¹, 2204 cm⁻¹, 1720 cm⁻¹. The ¹H NMR (CDCl₃) showed signals at δ 9.2(s,1H), 8.6(1H,d), 7.8(d,2H), 7.4(d,1H), 4.4(q,2H), 3.1(dd,2H), 2.0(dd,2H), 1.5-1.6(t,3H), 1.2-1.4(s,6H). The CI Mass showed M+. m/z 352.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

What is claimed is:

1. A process for the preparation of a disubstituted acetylene bearing heteroaromatic and heterobicyclic groups of formula I 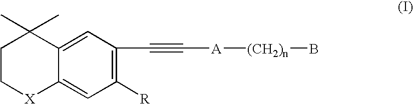
wherein X is S, O, or NR¹ wherein R¹ is hydrogen or a C₁-C₆ straight or branched alkyl group; R is hydrogen or a C₁-C₆ straight or branched alkyl group; A is a substituted or unsubstituted pyridinyl, thienyl, furyl, pyridazinyl, pyrimidinyl or pyrazinyl group; n is 0-4; and B is H, —COOH or a pharmaceutically acceptable salt thereof or an amide or a mono or di-substituted amide thereof, —CH₂OH, —CHO or a C₁-C₆ alkyl acetal derivative, —COR² or a C₁-C₆ alkyl ketal derivative wherein R² is —(CH₂)ₘCH₃ wherein m is 0-4 or COOR³ wherein R³ is a straight or branched C₁-C₃₀ alkyl group, a substituted or unsubstituted C₆-C₃₀ aromatic group, a substituted or unsubstituted C₃-C₃₀ cycloalkyl, a substituted or unsubstituted C₃-C₃₀ cycloalkylalkyl, a substituted or unsubstituted C₃-C₃₀ cycloalkenyl, a substituted or unsubstituted C₅-C₃₀ aryl, a substituted or unsubstituted C₅-C₃₀ arylalkyl, a substituted or unsubstituted C₅-C₃₀ heteroaryl, a substituted or unsubstituted C₃-C₃₀ heterocyclic ring, a substituted or unsubstituted C₄-C₃₀ heterocyclylalkyl, a substituted or unsubstituted C₆-C₃₀ heteroarylalkyl, the process comprising a Sonogashira coupling reaction between a compound of formula II 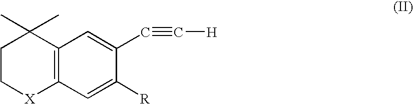
wherein X and R have the aforestated meanings, with a compound of formula III
X′-A-(CH₂)ₙ—B (III)
wherein X′ is a halogen and A, n and B have the aforestated meanings, in the presence of a base and a transition metal catalyst and in a polar aprotic solvent.

2. The process of claim 1, where in the compound of formula II X is S and R is hydrogen, and in the compound of formula III A is pyridyl, thienyl or furyl, and n is 0 or 1.

3. The process of claim 1, where in the compound of formula II X is S and R is hydrogen, and in the compound of formula III A is pyridyl, thienyl or furyl, n is 0 or 1 and B is COOH or a pharmaceutically acceptable salt, lower alkyl ester or mono or di-lower alkyl amide thereof.

4. The process of claim 1, where in the compound of formula II X is S and R is hydrogen, and in the compound of formula III A is pyridyl, thienyl or furyl, n is 0 and B is COOR³ wherein R³ is a straight or branched C₁-C₆ alkyl group.

5. The process of claim 1, where in the compound of formula II X is S and R is hydrogen, and in the compound of formula III A is pyridyl, n is 0 and B is COOR³ wherein R³ is a straight or branched C₁-C₆ alkyl group.

6. The process of claim 1, wherein the compound of formula II is 4,4-dimethyl-6-ethynylthiochroman and the compound of formula III is ethyl-6-chloro-3-nicotinate.

7. The process of claim 1, wherein the base is selected from the group consisting of an alkali metal carbonate, alkali metal bicarbonate, alkali metal hydride, alkali metal hydroxide, alkali metal alkoxide, organic amine and mixtures thereof.

8. The process of claim 1, wherein the base is an organic amine selected from the group consisting of triethylamine, tributylamine, diethylamine, diisopropylethylamine, N-methylmorpholine, pyridine, 4-(N,N-dimethylamino)pyridine, N,N-dimethylaniline, N,N-diethylaniline, 1,5-diazabicyclo[4.3.0]nona-5-ene, 1,4-diazabicyclo[2.2.2]octane, 1,8-diazabicyclo[5.4.0]undec-7-ene and mixtures thereof.

9. The process of claim 1, wherein the transition metal catalyst is a palladium catalyst.

10. The process of claim 9, wherein the palladium catalyst is selected from the group consisting of palladium acetate, palladium chloride, palladium carbonate, bis(triphenylphosphine) palladium (II) chloride and mixtures thereof.

11. The process of claim 1, wherein the polar aprotic solvent is selected from the group consisting of a nitrile, an amide, a sulfoxide and mixtures thereof.

12. The process of claim 5, wherein the polar aprotic solvent is selected from the group consisting of a nitrile, an amide, a sulfoxide and mixtures thereof.

13. The process of claim 1, wherein the polar aprotic solvent is selected from the group consisting of dimethyl sulfoxide, dimethyl formamide, dimethyl acetamide and mixtures thereof.

14. The process of claim 1, wherein the polar aprotic solvent is present in an amount of about 5 volumes to about 15 volumes.

15. The process of claim 1, wherein the polar aprotic solvent is present in an amount of about 7 volumes to about 10 volumes.

16. The process of claim 1, further comprising a cuprous halide is selected from the group consisting of cuprous fluoride, cuprous chloride, cuprous bromide, cuprous iodide and mixtures thereof.

17. The process of claim 1, wherein the reaction is carried out at a temperature of about 20° C. to about 200° C.

18. The process of claim 1, comprising adding a solution containing the transition metal catalyst and polar aprotic solvent to a solution containing the base, the compound of formula II, and the compound of formula III and heating to a temperature of about 80° C. to about 110° C.

19. The process of claim 1, wherein the disubstituted acetylene bearing heteroaromatic and heterobicyclic groups of formula I is thereafter converted to a pharmaceutically acceptable salt thereof.

20. The process of claim 1, wherein the disubstituted acetylene bearing heteroaromatic and heterobicyclic groups of formula I is tazarotene.

21. The process of claim 1, further comprising the steps of:
adding an inorganic acid to the reaction mixture to provide a salt of the disubstituted acetylene of formula I;
adding an inorganic base to the salt of the disubstituted acetylene of formula I in a second solvent; and
isolating the disubstituted acetylene of formula I.

22. The process of claim 21, wherein the inorganic acid is selected from the group consisting of hydrobromic acid, hydrochloric acid, sulfuric acid, perchloric acid and phosphoric acid.

23. The process of claim 21, wherein the inorganic acid is present in a solution.

24. The process of claim 21, wherein the second solvent is ethyl acetate.

25. The process of claim 21, wherein the yield of the product disubstituted acetylene of formula I is at least about 65%.

26. The process of claim 21, wherein the yield of the product disubstituted acetylene of formula I is at least about 80%.

27. The process of claim 1, wherein the purity of the product disubstituted acetylene of formula I is at least about 95%.

28. The process of claim 1, wherein the purity of the product disubstituted acetylene of formula I is at least about 99.5%.

29. The process of claim 20, further comprising the steps:
adding an inorganic acid to the reaction mixture to provide a salt of tazarotene;
adding an inorganic base to the salt of tazarotene in a second solvent; and
isolating tazarotene.

30. The process of claim 29, wherein the purity of tazarotene is at least about 99.5%.

31. A process for the preparation of tazarotene comprising a Sonogashira coupling of 4,4-dimethyl-6-ethynylthiochroman with ethyl-6-chloropyridine-3-carboxylate in the presence of a base and a transition metal catalyst and in a polar aprotic solvent.

32. The process of claim 31, wherein the base is an organic amine selected from the group consisting of triethylamine, tributylamine, diethylamine, diisopropylethylamine, N-methylmorpholine, pyridine, 4-(N,N-dimethylamino)pyridine, N,N-dimethylaniline, N,N-diethylaniline, 1,5-diazabicyclo[4.3.0]nona-5-ene, 1,4-diazabicyclo[2.2.2]octane, 1,8-diazabicyclo[5.4.0]undec-7-ene and mixtures thereof.

33. The process of claim 31, wherein the transition metal catalyst is a palladium catalyst.

34. The process of claim 33, wherein the palladium catalyst is selected from the group consisting of palladium acetate, palladium chloride, palladium carbonate, bis(triphenylphosphine) palladium (II) chloride and mixtures thereof.

35. The process of claim 31, wherein the polar aprotic solvent is selected from the group consisting of a nitrile, an amide, a sulfoxide and mixtures thereof.

36. The process of claim 31, wherein the polar aprotic solvent is selected from the group consisting of dimethyl sulfoxide, dimethyl formamide, dimethyl acetamide and mixtures thereof.

37. The process of claim 31, further comprising a cuprous halide is selected from the group consisting of cuprous fluoride, cuprous chloride, cuprous bromide, cuprous iodide and mixtures thereof.
